(19) United States
(12) Patent Application Publication
(10) Pub. No.: US 2004/0158155 A1
(43) Pub. Date: Aug. 12, 2004

(54) Transcranial doppler spectroscopy for assessment of brain cognitive functions

(76) Inventor: Philip Chidi Njemanze, Owerri (NG)

Correspondence Address: PHILIP CHIDI NJEMANZE, NO 1 URATTA/MCC ROAD, P O BOX 302, OWERI, POB302, NG

(21) Appl. No.: 10/770,606

(22) Filed: Feb. 3, 2004

Related U.S. Application Data
(63) Continuation-in-part of application No. 10/109,867, filed on Apr. 1, 2002.

(51) Int. Cl. A61B 8/06

(52) U.S. Cl. 600/454

(57) ABSTRACT

A noninvasive method to determine cerebral blood flow velocity response to face recognition tasks of a human subject, including steps of obtaining a subject's cerebral blood flow velocity in cerebral arteries on both sides of the brain using a microcomputer integrated with a transcranial Doppler ultrasound instrument with two probes placed on the temples and sample volumes focused on cerebral vessels on both sides and calculating laterality index for both arteries. Simultaneously, testing the subject with face or object processing tasks presented on the screen of a digital computer while monitoring the mean blood flow velocity during each stage of the task in real-time. Processing the acquired data to determine the spectrum analysis using a microcomputer that is operatively connected to a computer workstation for image retrieval and cross matching.

### CROSS-REFERENCE TO RELATED APPLICATION

This application is related to and comprises a continuation-in-part of the patent application filed by the same inventor on Apr. 1, 2002, under Ser. No. 10/109,867.

### U.S. PATENT DOCUMENTS

U.S. Pat. No. 5,295,491 March 1994 Givens, Alan S. 600/544

U.S. Pat. No. 5,724,987 March 1998 Givens et al. 600/544

U.S. Pat. No. 5,771,261 June 1998 Anbar, Michael 374/45

U.S. Pat. No. 6,126,595 October 2000 Amano et al. 600/300

U.S. Pat. No. 6,309,361 October 2001 Thorton, Kirtley E. 600/544

U.S. Pat. No. 6,390,979 May 2002 Njemanze, Philip C. 600/438

U.S. Pat. No. 6,547,737 April 2003 Njemanze, Philip C. 600/454

U.S. Pat. No. 6,663,571 December 2003 Njemanze, Philip C. 600/504

### STATEMENT REGARDING FEDERALLY SPONSORED RESEARCH OR DEVELOPMENT

Not Applicable

### REFERENCE TO MICROFICHE APPENDIX

Not Applicable

### BACKGROUND OF THE INVENTION

This invention is related to use of transcranial Doppler ultrasound for determination of brain cognitive function, more specifically, the invention is related to computerized systems and methods for determining facial and object recognition, odor evaluation, computerized mental performance testing using a portable non-invasive transcranial Doppler equipment operatively connected to a microcomputer, and the system linked to a computer workstation.

The application of the present invention cover assessment of human cognition, primate and canine processing of psychophysiologic functions. However, the applications of the present invention would be demonstrated using face and object recognition tasks by way of example only.

Face and object recognition is becoming increasingly important to identify criminal suspects, however, the neurophysiology is still poorly understood. With the growing threat of terrorism and in the aftermath of the Sep. 11, 2001 terrorist attack on the twin Towers of the World Trade Center in New York, recognition of the faces of terrorist suspects entering the airports, seaports and border crossings is crucial to National Security. However, biometric methods introduced to improve security are static, mainly based on cross matching the face of the traveler with that in the international passport and fingerprints. While this maybe an improvement to prior situation of total absence of biometric information, its importance in crime prevention may be limited. However, it may serve to provide a database of biometric information including faces and fingerprints. In its present form, the data is static and would not, for example, identify suspects with cosmetic or plastic surgery modification of their faces to escape identification. However, it is possible to train persons that could be referred to as ‘face-minders’, to memorized faces of suspects on terrorist watch-list, by way of example. The immigration officer trained as a ‘face-minder’ is here referred to as ‘immigration officer and face-minder’. Trainees could acquire skills of cross-matching key features of faces of persons seen at the ports as compared to that in the immigration or forensic facial database. However to be effective, subjective judgment must be replaced with objective physiologic correlates of good matches. This will require objective online detection of physiologic variables, suggestive of facial memory involvement and cross matching the online variables to expected variables, for the particular face involved. Online brain imaging adapted for use for such applications include electrophysiological techniques and transcranial Doppler ultrasound.

Electrophysiological devices particularly the electroencephalography (EEG) has been used to determine the state of mental performance in general. The patents, U.S. Pat. No. 5,295,491 to Givens and U.S. Pat. No. 5,724,987 to Givens et al, described a method and system for testing the mental performance capability of a human subject, which includes, a digital computer workstation, for presenting a test to the subject, such as visuomotor memory task. U.S. Pat. No. 6,126,595 to Amano et al. teaches a device for diagnosing physiological state, based on blood pulse waves detected in the body. U.S. Pat. No. 6,258,032 B1 to Anbar, teaches a method for assessment of the effects of mental stress, involving the measurement of periodic changes in skin perfusion. U.S. Pat. No. 6,309,361 B1 to Thorton, teaches a method for improving memory by identifying and using EEG parameters correlated to specific cognitive functioning, wherein, the cognitive abilities addressed include, memory for auditory and visual (face, Korean characters, reading material). U.S. Pat. No. 6,390,979 to Njemanze, described a noninvasive transcranial Doppler ultrasound computerized mental performance testing system. The device assesses multi-modality related working memory and communicates the outcome to an operatively connected computer. U.S. Pat. No. 6,663,571 B1 to Njemanze, described a noninvasive transcranial Doppler ultrasound computerized odor evaluation testing system, for odor matching and odor selection in canine and human subjects.

Transcranial Doppler (TCD) sonography is an ultrasound technique that uses Doppler principles to measure cerebral blood flow velocity in major brain arteries of the circle of Willis. The basic principles and common clinical applications are detailed in a book edited by Aaslid R, entitled “Transcranial Doppler Sonography” and published by Springer, of Wien, N.Y., dated 1989, on pages 39 through 50. Transcranial Doppler could be used to assess brain psychophysiologic functions, including language as disclosed by Njemanze P C, in an article entitled “Cerebral lateralizaion in linguistic and nonlinguistic perception: analysis of cognitive styles in the auditory modality”, published in Brain and Language, dated 1991, volume 41, pages 367 through 380; colors, as disclosed by Njemanze et al., in an article entitled “Cereoral lateralization and color perception: a transcranial Doppler Study” published in Cortex, dated 1992, volume 28, pages 69 through 75; letter processing, as disclosed by Njemanze P C, in an article entitled “Cerebral lateralization in random letter task in the visual modality: a transcranial Doppler study”, published in Brain and Language, dated 1996, volume 63, pages 315 through 325; music, as disclosed by Evers et al, in an article entitled “The cerebral hemodynamics of music perception. A transcranial Doppler sonography study”, published in Brain, dated 1999 volume 122, pages 75 through 85; and a variety of mental functions, as disclosed by Vingerhoets & Stroobant, in an article entitled “Lateralization of cerebral blood flow velocity changes during cognitive tasks. A simultaneous bilateral transcranial Doppler study”, published in Stroke, dated 1999, volume 30, pages 2152 through 2158. Studies with TCD have been cross validated by functional MRI, as disclosed by Schmidt et al., in an article entitled “Determination of cognitive hemispheric lateralization by functional transcranial Doppler cross-validated by functional MRI”, Stroke, dated 1999, volume 30, pages 939 through 945, and reproducibility assessed, as described by Knecht et al., “Reproducibility of functional transcranial Doppler sonography for determining hemispheric language lateralization”, published in Stroke, dated 1998, volume 29, pages 1155 through 1159.

### BRIEF SUMMARY OF THE INVENTION

This invention is related to use of transcranial Doppler ultrasound for determination of brain cognitive function, more specifically, the invention is related to computerized systems and methods for determining facial and object recognition, odor evaluation, computerized mental performance testing using a portable non-invasive transcranial Doppler equipment operatively connected to a microcomputer, and the system linked to a computer workstation.

Until now, transcranial Doppler assessment of brain cognitive function, has been based on mean blood flow velocity changes. The use of spectrum analysis has not been applied in the assessment of psychophysiologic brain function. The use of mean flow velocity as a psychometric index is subject to pitfalls, including probe-vessel angle variations, respiratory artifacts related to end-tidal partial pressure of carbon dioxide oscillations, and motion induced artifacts. One advantage of the present invention is the use of the spectrum analysis from Fourier series, that examines the cyclical pattern of data in response to cognitive stimuli. The classic description of spectrum analysis is given by Bloomfield P, in a book entitled “Fourier analysis of time series: an introduction”, published in New York, by Wiley, dated 1976. The purpose of the analysis, is to decompose a complex time series with cyclical components into a few underlying sinusoidal (sine and cosine) functions of particular wavelengths.

The wavelength of a sine or cosine function is typically expressed in terms of the number of cycles per unit time (frequency), often denoted by ƒ. The period of the sine or cosine function is defined as the length of time required for one full cycle. Thus, it is the reciprocal of the frequency, or: T=1/ƒ. The sine and cosine functions are mutually independent (or orthogonal); thus you may sum the squared coefficients of each frequency to obtain the periodogram. Specifically the periodogram values are computed as:

Pₖ=sine coeff.ₖ₂*cosine coeff.ₖ₂*n/2;

where Pₖ is the periodogram value of frequency ƒₖ and n is the overall length of the series. The spectral densities are the frequency regions, consisting of many adjacent frequencies, which contribute most to the overall periodic behavior of the series. This can be accomplished by smoothing the periodogram values via a weighted moving average transformation.

To assess hemispheric lateralization, there is need to uncover the correlation between two series derived from both hemispheres respectively, at different frequencies. This is called cross-spectrum analysis. However, the cross-spectrum consists of complex numbers that can be divided into a real and an imaginary part. These can be smoothed to obtain the cross-density and quadrature density (quad density for short) estimates, respectively. The square root of the sum of the squared cross-density and quad-density values is called the cross-amplitude. The cross-amplitude can be interpreted as a measure of covariance between the respective frequency components in the two series. In order, to obtain standardized cross-amplitude values, one could square them and divide by the product of the spectrum density estimates for each series. The result is called the squared coherency, which can be interpreted similarly to the squared correlation coefficient in basic statistics.

The phase shift estimated (φ), is a measure of the extent to which each frequency component of one series leads the other. The use of transcranial Doppler measurement of mean blood flow velocity in the cerebral arteries and the spectrum analysis derived from these cyclical oscillations recorded in response to behavioral, cognitive or other stimuli or conditions, is hereby referred to as transcranial Doppler (TCD) ‘spectroscopy’. The present invention describes by way of example, the application of TCD spectroscopy in cognitive biometrics. Thus, the ‘cognitive biometrics’ refers to use of brain cognitive function as a marker, for biometric characterization of a subject under study.

The special embodiment of this invention is illustrated in the specification, it includes block diagram for the format of the instrumentation, and how the system functions is shown by way of example, at an immigration port of entry. The human subject whose face is being studied is hereby referred to as ‘immigrant’. However, the ‘immigrant’ category also covers persons with non-immigrant visas. The human studying the face and whose perception is being analyzed is hereby referred to as the immigration officer and ‘face-minder’. The face-minder is trained to memorize the faces and key facial features of suspected criminals or terrorists. The personnel observing both the immigrant and immigration officer and face-minder is hereby referred to as the ‘operator’. The immigration officer and face-minder is interfaced with the system, by way of placing two 2 MHz transducers in a headgear, directed on the temporal bone. A detail of placement and the settings for such a headgear has been disclosed in U.S. Pat. No. 6,547,737 to Njemanze. The right (RMCA) and left (LMCA) middle cerebral arteries supply 80% of the brain regions respectively, and could be insonated from both sides. The mean cerebral blood flow velocity (MBFV) is recorded. The side-to-side difference or laterality index (LI) is assessed, to determine the hemisphere activated. When for example, the left hemisphere is activated in an immigration officer and face-minder, it could be presumed that the face of the immigrant has been seen before, and a thorough search within the facial database is made to obtain matches. Matches could be that of a prior visitor or a criminal suspect. It has been disclosed in U.S. pat. application Ser. No. 10/109,867 by Njemanze, that faces that evoke negative emotions (for example, sad face) differ significantly in lateralization from those that evoke positive emotions (for example, happy face). The spectrum analysis associated with both faces are matched to that of the face viewed in real-time. The face of terrorists and criminal suspect would be expected to induce greater left lateralization and higher spectral density peaks in the left hemisphere.

Prior art of cognitive assessment, has not implemented the use of neuroimaging methods in particular transcranial Doppler in real-time life situations, for assessment of facial stimuli for forensic and law enforcement purposes. The present invention illustrates by way of example, the use of transcranial Doppler for facial analysis suitable for application in immigration services.

All the existing systems of ascertaining facial identity rely on using pictures of the suspect and comparing with that in the database. Changes in the presenting face, relative to that in the database are usually difficult to detect by automated systems, especially with increasing sophistication of facial reconstructive plastic surgery. In other words, present techniques compare static images and are totally ineffective when changes in facial attributes are introduced by way of plastic surgery or using other manners of disguise. However, humans despite changes in facial feature, could be trained to recognize faces using key attributes. It is therefore desirable to have a system of human-computer interface in facial recognition tasks.

Although, human recognition of faces is effective in determining the identity of criminals, false positive recognition could become very embarrassing to innocent persons and to authorities. Often, immigration officials might not express near recognition of someone as a precaution to avoid embarrassing errors. This situation in some cases, could allow dangerous criminals to escape without being stopped. In others, innocent people might face embarrassing situations, causing airlines to cancel scheduled flights or even airports to be shut down for fear of persons thought to be would-be terrorists. Similar situations have arisen, leading to cancellation of flights by British Airways and Air France in t December, 2003. Los Angeles Airport in December, 2003 was shut down for several hours, for similar reasons. These events cost several millions of dollars to the airlines and airport authorities. What is therefore required is a system that assesses brain response of the immigration officer to the face of the immigrant, and comparison of the immigrant face to that in the database, without expression of any suspicion except when a positive match is made.

An advantage of the present invention is to provide a system, that triggers fast restricted search of a face in a facial database prompted by human recognition of some key attributes of the suspect face.

An advantage of the present invention is to provide a system, that uses the evoked physiological response in the brain of a human to key facial attributes to trigger a detailed database search for a match.

An advantage of the present invention is that, it would be less vulnerable to errors of recognition due to facial makeup or disguise.

Another object of this invention is that it allows better integration of multi-sensory biometric measures, including those related to human facial recognition, computerized facial recognition and overall human perception of the state-of-being of the suspect, as well as cross-matching with other biometric measures such as fingerprints.

Another object of the present invention is to introduce the use of cognitive biometric systems that identifies individuals based on objective psychophysiologic brain responses to given attributes, which may comprise of singular (visual, auditory, tactile, olfactory) or multimodality sensory responses.

It is an object of the present invention, to use cognitive biometric systems to identify individuals or related objects based on odor evaluation.

It is an object of the present invention, to use cognitive biometric systems to identify individuals by the mental performance on a predefined task.

It is an object of the present invention, to use cognitive biometric systems to identify objects by their colors.

Another object of the present invention, is the use of cognitive biometrics for dynamic real-time assessment of subject's identification profile in a geographically referenced environment.

Until now, none of the prior art, alone or in combination teaches or suggests a noninvasive method to determine the cerebral blood flow velocity response to face recognition task of a human subject including the steps of obtaining a subject's baseline cerebral blood flow velocity using a transcranial Doppler instrument with sample volumes focused on cerebral vessels on both sides using two probes placed on the temples and calculating laterality index for both arteries, and using the velocity trend recorded for a given time, to perform a spectrum analysis, and subsequently, testing the subject with live presentations of faces of individuals, while simultaneously monitoring the mean blood flow velocity during each stage of the task in real-time, and then comparing the online spectrum analysis to that stored in a portable transcranial Doppler, operatively connected to a pocket personal computer (PC) or handheld PDA, while prompting that, an operator using a computer input peripheral device at a main frame computer terminal performs a similar facial recognition task.

These and other objects, may become more apparent to those skilled in the art, upon reviewing the description of the invention, as set forth hereinafter, in view of its drawings.

### BRIEF DESCRIPTION OF THE DRAWINGS

FIG. 1a shows a diagram of the invention adapted for use at an immigration entry port;

FIG. 1b shows the diagram of the remote operator of the computer database;

FIG. 1c shows in detail the invention adapted for use by an immigration officer;

FIG. 2 shows the screen of the device with acquired data;

FIG. 3 shows the screen display of the device in use;

FIG. 4 shows the images that were presented to the experimental subjects;

FIG. 5 shows the spectral density plots in male and female subjects in the RMCA and LMCA respectively;

FIG. 6 shows the cross amplitude for the males and females respectively;

FIG. 7 shows the squared coherence plots in males and females subjects;

FIG. 8 shows the schematic diagram of one embodiment of the invention;

FIG. 9 shows the flow chart of one embodiment of the invention.

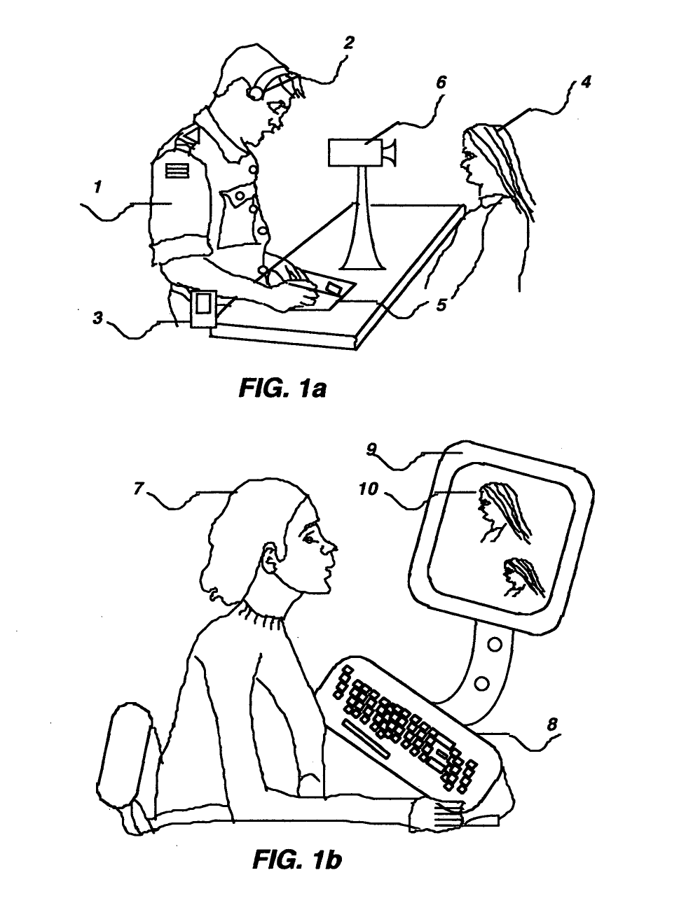

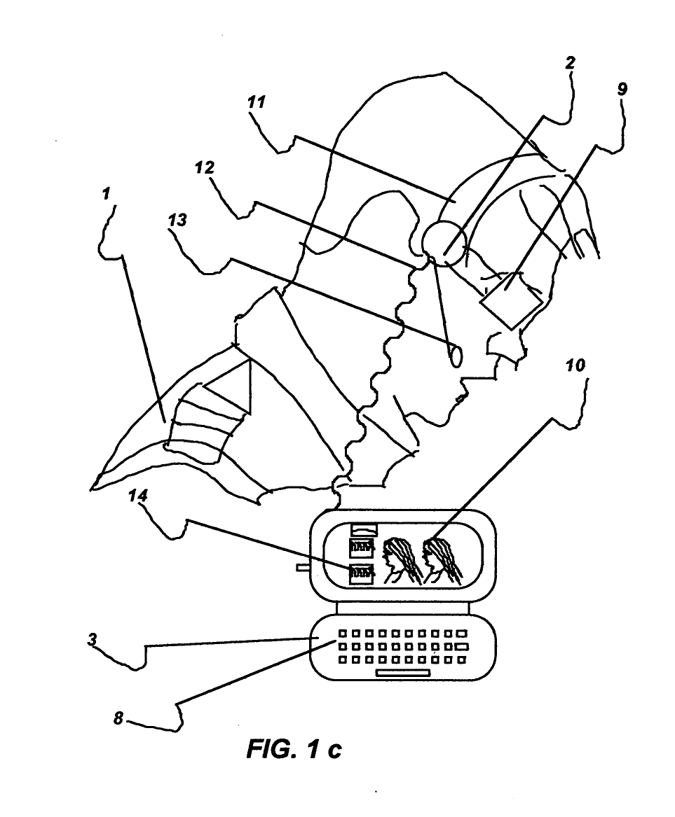

FIG. 1a shows a diagram of the invention adapted for use at an immigration entry port. The immigration officer and face-minder 1, is interfaced with two ultrasound transducers 2 fitted in a headgear on both sides. Details of placement and the settings for such a headgear have been disclosed in U.S. Pat. No. 6,547,737 to Njemanze. The transcranial Doppler equipment components could be obtained from DWL, Singen, Germany, and integrated into a pocket PC or handheld PDA device, such as that commercially available from Hewlett-Packard Company, Palo Alto, Calif. 3, such a portable device could be attached to the belt or put in the pocket of the immigration officer and face-minder as a carry-on device, by way of example. The immigration officer face-minder 1 triggers the device 3 by remote control button, voice activation or simply pressing the data acquisition button on the surface panel. The data acquisition starts as the face-minder concentrates to study the face of the immigrant 4 for about 30 seconds. And thereafter the immigration officer studies the passport 5 and other formalities. During this time the MBFV in both the RMCA and LMCA are acquired and used for further processing. The face of the immigrant is captured by means of a camera 6.

FIG. 1b shows the diagram of the remote operator of the computer database. The camera 6 allows the remote operator (preferred female operator) 7 working at a computer terminal 8 to observe on a monitor 9 the face of the immigrant 10. The operator 7 could retrieve from the database, the face 10 of the immigrant, and related record stored in the database for comparison. The remote computer 8, also receives data from the device 3, on the brain response of the face-minder 1.

FIG. 1c shows in detail the invention adapted for use by an immigration officer. Several accessories could be attached to the head gear 11, which houses the ultrasound transducers 2, and connected to the portable microcomputer 3, by means of a cable 12. These accessories may include a microphone13 for voice activated control of the device. The images including cerebral blood flow data 14 appear on the monitor 9 of the device, and also displayed on a visor or eye-piece monitor 9, that flips into view of the immigration officer, allowing visualization of the retrieved face from the database, personal records and spectral analysis plots. Such eye-piece monitors could be adapted from commercially available systems, such as Eye-Trek® from Olympus Optical Corporation GmbH, Hamburg, Germany. There could be also a text-based communication with the operator 7, via the visual display on the visor. The device has also a keyboard 8, and capability for wireless communication.

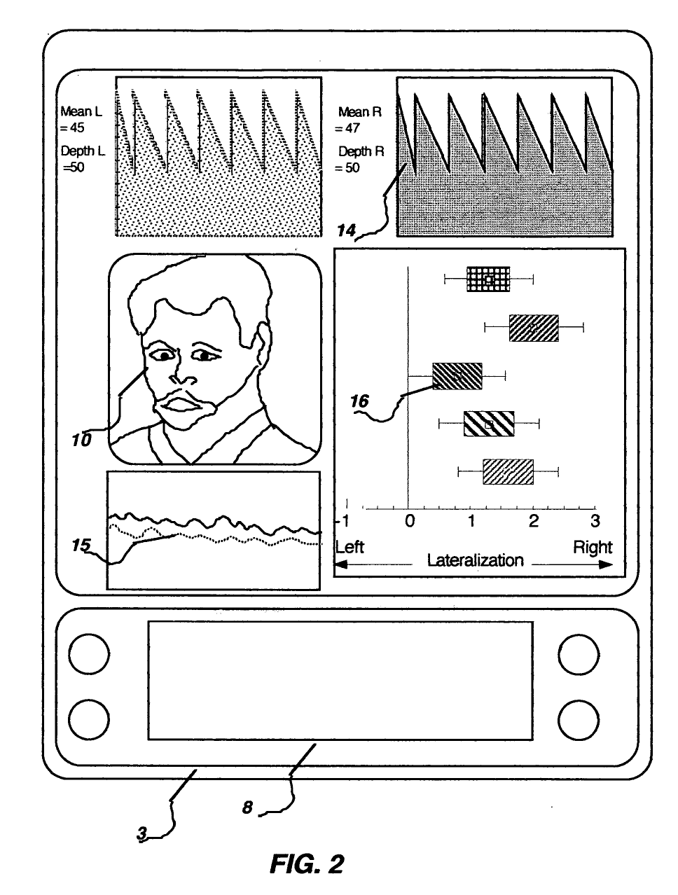

FIG. 2 shows the screen of the device with acquired data. The data acquired by the device 3, include the immigrant face 10, the MBFV in the LMCA and RMCA 14, the trend velocities in both arteries 15. After initial processing, the device displays the lateralization pattern 16 for the face with reference to other faces. The device settings could be changed using a keyboard 8. A relative left lateralization, denotes left hemisphere activation implicating memory. This suggests that the face-minder has seen the face before and thus might be related to that within the database. Enhanced activity in the left hemisphere has been seen with faces associated with negative emotions, as demonstrated in U.S. pat. application Ser. No. 10/109,867, and disclosed by Morris et al., in an article entitled “A modulatory role of the human amygdala in processing emotional facial expressions”, published in Brain, dated 1998, volume 121, pages 47-57. Conversely, a greater right hemisphere activation was seen with faces associated with positive emotions, as described by Gorno-Tempini et al., in an article entitled “Explicit and incidental facial expression processing: an fMRI study”, published in Neuroimage, dated 2001, volume 14, pages 465-473. The immigration officer and face-minder undergoes basic training and updates, as often as possible, even within a matter of days. For example, in the United States, where acquisition of facial database has been implemented along with fingerprints, it could be possible to accumulate a large facial database in a relatively short time. All faces acquired from the immigration entry ports into the United States could be stored in a database accessible from any immigration entry port or border crossing point. With more countries developing similar databases, an online secure Internet link could be established between Europe, Japan and USA, by way of example. The faces of dangerous criminals and terrorists along with horror scenes of their associated activities could be shown to face-minders and updated regularly. This will pose a considerable task for the immigration officer to memorize each face in detail. However, the brain will respond differently when presented with a novel facial stimuli, as compared to a repeated stimulation with the same or similar face, and more so when attached to negative emotions. This ability of the brain memory function in practice is unlimited. The cerebral lateralization and brain spectrum analysis (see below for details) response evoked by the face of a wanted criminal is stored in the portable device for each face-minder. This response pattern is unique for each face, and reproducible on stimulation. On perception of a face at the border crossing, the immigration officer and face-minder could activate the system to search its database for a biometric match. Some PDA devices such a those from Hewlett Packard Company, Palo Alto, Calif., already store some biometric fingerprint data. Such PDAs could be used for this purpose, otherwise pocket PC devices could be adapted for this purpose. The present invention could integrate the spectrum analysis, facial information, the fingerprint, and personal records of the immigrant, for use in cross matching real-time to archival data. A positive match, could trigger a more thorough review by the operator at the computer terminal. Based on corroborative positive match, the necessary measures could be initiated. This method could significantly decrease the amount of false-positive identifications, that has become a source of costly terror alerts around the World. The choice of males as face-minders and females as operators would become obvious, on review of the results of an experiment performed by the inventor. A detailed description of this experiment to determine LI and spectrum analysis, is set forth below.

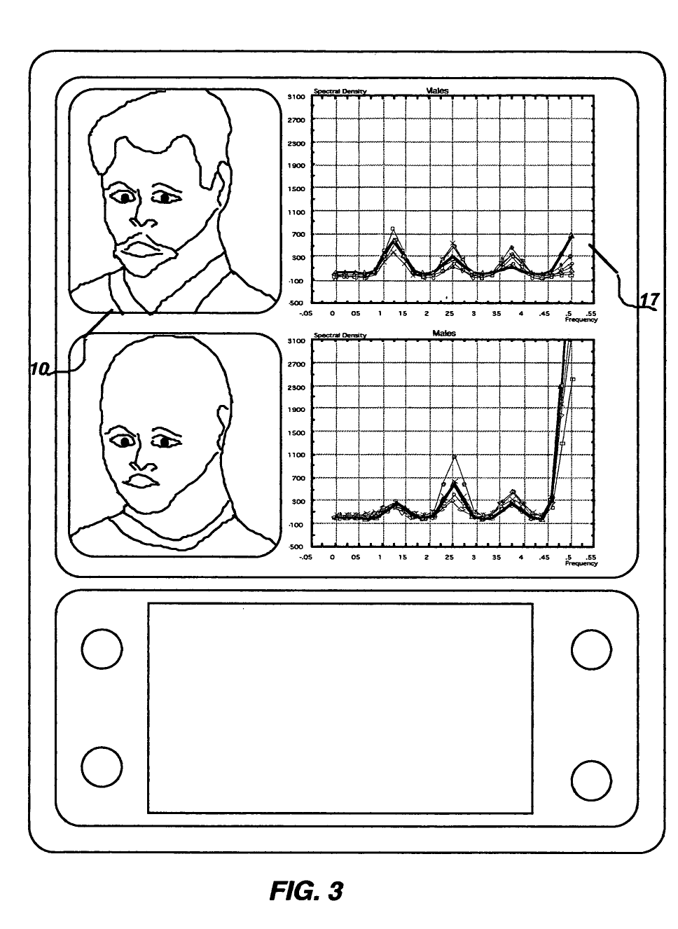

FIG. 3 shows the screen display of the device in use. The images of the faces are matched to their spectral densities 17, as determined below.

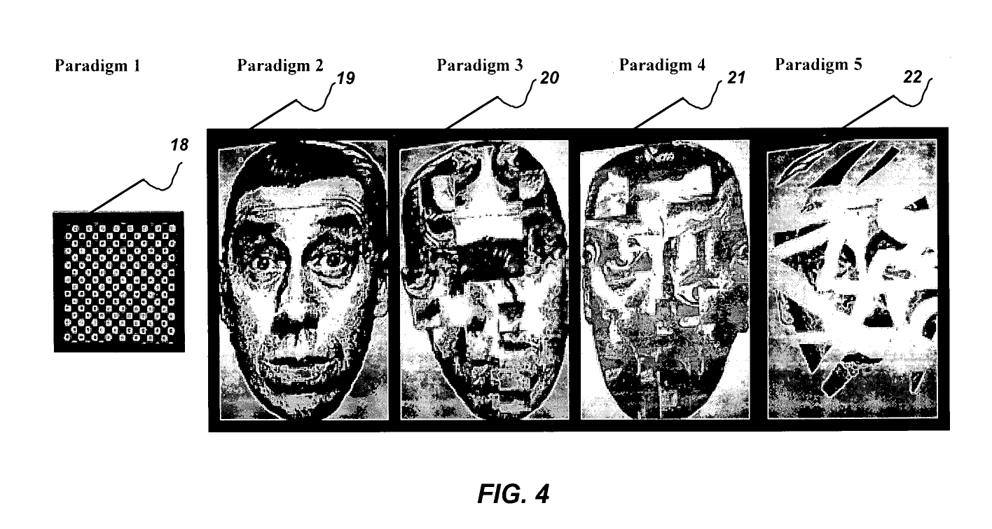

FIG. 4 shows the images that were presented to the experimental subjects. The object 18 was presented as control condition Paradigm 1, to compare to faces presented in experimental conditions. The facial tasks included a whole neutral face Paradigm 2 19, a facial working memory task Paradigm 3, 20 facial recognition tasks Paradigm 4 21, and Paradigm 5 22.

Methods

Simultaneous bilateral TCD ultrasound was used to measure MBFV in the RMCA and LMCA in right-handed normal subjects. The preferred hand was determined using the Edinburgh handedness inventory (Oldfield, 1971). The population consisted of 16 subjects, (mean±SD age=24.8±2.7 years) of which 8 females were age matched to 8 males. Subjects were matched by anthropometric variables. Body mass index (BMI) was calculated as weight/height squared (in Kg/m₂), (for males was 23±2 Kg/m₂ and females was 23±5 Kg/m₂); and waist-to-hip ratio (WHR) (for males was 0.85±0.03 and females was 0.8±0.05). All subjects had normal blood pressure, systolic blood pressure (for males was 110±6 mmHg and females was 107±5 mmHg), and diastolic blood pressure (for males was 70±6 mmHg and females was 70±9 mmHg). All subjects had normal visual acuity. None reported any history of neurological or cardiovascular or respiratory diseases. Subjects were not under any medication, including contraceptive pills for females, during the time of the study. All were non-smokers and there was no report of alcohol abuse in subjects and their immediate families. None ingested caffeine at least 24 hours prior to the study. All have had 16-18 years of schooling. All subjects signed informed consent according to the Declaration of Helsinki, and the Institutional Review Board approved the study protocol.

Scanning Procedure

All TCD procedures were performed using examination techniques previously described for cognitive studies as disclosed in works, by Njemanze et al., in an article entitled “Cerebral lateralization and color perception: a transcranial Doppler Study”, published in Cortex, dated 1992, volume 28, pages 69 through 75; by Njemanze P C, in an article entitled “Cerebral lateralzation in random letter task in the visual modality: a transcranial Doppler study”, published in Brain and Language, dated 1996, volume 63, pages 315 through 325; by Wittich et al., in an article entitled “Visually evoked perfusion changes in the posterior cerebral artery during activation of various visual field sections”, in a book edited by J. Klingenhofer, et al., entitled “New Trends in Cerebral Hemodynamics and Neurosonology” published by Elsevier of Amsterdam, dated 1997, on pages 548-556; by Vingerhoets G, and Stroobant N, in an article entitled “Lateralization of cerebral blood flow velocity changes during cognitive task. A simultaneous bilateral transcranial Doppler study”, published in Stroke, dated 1999, volume 30, pages 2152-2158; by Stroobant N, and Vingerhoets G, in an article entitled “Transcranial Doppler ultrasonography monitoring of cerebral hemodynamics during performance of cognitive tasks: A review”, published in Neuropsychology Review, dated 2000, volume 10, pages 213-231. The TCD scanning was performed using bilateral simultaneous TCD instrument (Multi-Dop. T, DWL, Sipplingen, Germany). All subjects were briefed on the protocol for the entire experiment. All questions and practice sessions on what was required for the facial paradigm were explained prior to start of the experimental data acquisition. All TCD studies were performed as follows: first, the subject was placed in supine posture with head up at 30 degrees. The probe holder headgear—LAM Rack was used with base support on two earplugs and on the nasal ridge. Two 2 MHz probes were affixed in the probe holder, and insonation performed to determine the optimal position for continuous insonation of both MCA main stems at 50 mm depth from the surface of the probe. All gain and power settings were kept constant for both MCAs in all subjects. The probes were placed firmly on the subject's head and were locked in position after adjusting the lever and tightening the knurled screw from both sides. Comfort of the subject within the headgear was assured prior to start of recording. Subjects were instructed to remain mute and not to move throughout the data acquisition time duration, and informed that they would be debriefed of all mental activities after the experimental data acquisition session. All subjects were requested to refrain from internal or external verbalization, and informed of the deleterious effects it might have on the data acquired. All environmental noise, including sound from the TCD instrument was excluded. Environmental luminance was kept constant for all participants. Electrocardiographic monitoring of pulse and respiratory rate along with self perceived anxiety levels were recorded during the study to control for effects on results. Post-experimental debriefing focused on what subjects were “thinking” during task performance. Subjects were encouraged to provide full disclosure of their thoughts during the experimental data acquisition. No threat of penalty was made for non-compliance. Pre-experimental test runs in a different but selected group of subjects provided insights to how subjects handled the stimulus used in the present study. This was taken into account in the task design of the present study.

Baseline Recording

The baseline condition was dark. This was achieved by having the subject view through a 3-D View-Master, International group Inc. Portland, Oreg. with all its inside walls colored with black paint, and the back view covered with a dark slide. As a result the subject had full binocular vision with attention focused on a dark background. Side flaps shielded low intensity room light. Sound shielding of experimental room was provided and the subject wore earplugs that in addition reduced environmental noise levels. However, no subject was completely sensory (visual or auditory) deprived. A continuous train of velocity waveform envelopes was recorded at resting baseline with the subject mute, still and attention focused within a dark visual field, with no mental or manual tasks to perform. These baseline recordings were obtained prior to stimuli administration. TCD baseline measurements were made for 60 seconds. The same environmental conditions at baseline were maintained for data acquisition during each task. All tasks were designed to lack verbalizable features.

Paradigm 1: Checkerboard Square (Object Perception).

The black and white checkered square paradigm (18 FIG. 4) comprised a square of alternating black and white square dots. This was a nonverbal passive viewing task, of a foveally presented object, from a slide projector onto a screen, placed in front of the subject, and inclined at 30 degrees from the horizontal plane, at a distance of 80 cm from the nasal ridge. A continuous train of velocity waveform envelopes was recorded with the subject mute, still with fixed gaze and attention focused on the object. There were no mental or manual tasks to perform while viewing the object. MBFV baseline measurements were made for 60 seconds. All subsequent tasks presentations and recordings were performed in the same way.

Paradigm 2: Face Encoding Task (Whole Neutral Face)

This was a face-encoding task. A novel male neutral whole face (19 FIG. 4) was presented for a total of 60 seconds. The subject was instructed to commit the face to memory and told that their memory would be tested later. The MBFV was recorded during this 60 seconds as the subject viewed the face.

Paradigm 3: Facial Working Memory Task

This facial task comprised sorting elements of a disarranged face (20 FIG. 4). Subjects were asked to sort the elements of the face and arrange them into a whole face, one element at a time for duration of 60 seconds. The task required a sophisticated perceptual mechanism capable of extraction of components of a face, analysis of their width and height, distances between these elements, angles, contours, illumination, expression, hairline, hair style and so on, and constantly spatially fitting the puzzle by matching each element, with that stored in memory, and then proceeding to form the picture of the whole face. In other words, far more iterations are required to accomplish the recognition task. This facial paradigm was in effect a facial working memory task, and appears to require more activities in the visual associative cortex than just facial recognition. The MBFV was recorded for 60 seconds during the performance of the task.

Paradigm 4: Facial Recognition Task

This was a facial recognition task and comprised disarranged facial elements with a part of the face left in place as a clue (21 FIG. 4). Subjects were asked to recognize the face. The clue was intended to introduce some measure of ‘automaticity’ in the recognition process. In other words, the clue reduced the number of iterations required to accomplish the task of facial recognition. MBFV was recorded for 60 seconds during the performance of this task.

Paradigm 5: Facial Recognition Task

This was a facial recognition task with the face degraded with missing elements but the overall contour preserved (22 FIG. 4). The subject was required to recognize the face. MBFV was recorded for 60 seconds during the task.

For all tasks, the strategy and self-rated performance by subjects was ascertained during post-study debriefings.

Calculations

During analysis, artifacts during recording were marked and removed. Data averaging comprised 10 seconds segments of the train of velocity waveform envelopes for dark resting baseline, and each of the paradigms (1-5) respectively. Sixty seconds recording for baseline and each stimulus, resulted in six MBFV values for rest and each task respectively. These values were used for further calculations. Cerebral lateralization was assessed using laterality index (LI) expressed as:

LI=(RMCA MBFV_{10sec} minus LMCA MBFV_{10sec}/(RMCA MBFV_{10sec} plus LMCA MBFV_{10sec}))*100.

The actual magnitude of lateralization (LI) for each 10 seconds segment for each paradigm was calculated as the difference between LI values measured during the 10 seconds segment of the task and the corresponding 10 seconds segment of baseline (onset of resting baseline corresponds with onset of visual paradigm within the 60 seconds segment):

LI=LIparadigm_{10sec} minus LIbaseline_{10sec.}

In general, positive LI values suggest right lateralization, while negative LI values suggest left lateralization. Zero LI values showed no lateralization from the resting condition or possible bilateral response. The calculated LI were designated as LI_{0-10sec}, LI_{11-20sec}, LI_{21-30sec}, LI_{31-40sec}, LI_{41-50sec}, LI_{51-60sec}, for values measured for the time segment 0-10 seconds, 11-20 seconds, 21-30 seconds, 31-40 seconds, 41-50 seconds, 51-60 seconds respectively.

TCD Spectroscopy Method

To measure the frequency response characteristics of MBFV values in both RMCA and LMCA, fast Fourier transform algorithm (Statistica, StatSoft, OK, USA) was applied for all 48 _l data points in males and females respectively. First, the frequencies with the greatest spectral densities; that is the frequency regions, consisting of many adjacent frequencies, that contribute most to the overall periodic behavior of the series obtained in each vessel (RMCA and LMCA respectively), were determined and plotted. The Hamming window was applied as a smoother. The frequency peak, where the effects of dark baseline conditions were absent was designated as the stimulus spectral (S) peak. On the other hand, the mnemonic (M) peak was designated as that, which occurred in conjunction with dark and facial activation tasks. Cross spectrum analysis was undertaken to uncover the correlation between MBFV in the RMCA and LMCA at different frequencies. The cross amplitude was computed as the square root of the sum of the squared cross-density and quad-density values. The cross-amplitude was interpreted as a measure of covariance between the respective frequency components in the RMCA and LMCA. Squaring them and dividing by the product of the spectrum density estimates for each artery, standardized the cross-amplitude. The result called squared coherency, was interpreted as the squared correlation coefficient. The presence of large spectral density estimates for both RMCA and LMCA, and the cross-amplitude values at frequencies for S and M peaks suggest two strong synchronized periodicities in both series at those frequencies. The coherency value for the stimulus spectral peak S was used in further analysis. The phase shift estimates (φ) were computed as tan₋₁ of the ratio of the quad density estimates over the cross-density estimate. The phase shift estimates are measures of the extent to which each frequency component of one series leads the other.

Statistics

Results were given as mean±SD or mean±SE, where applicable. A 4-way analysis of covariance (ANCOVA) for repeated measures was performed. Gender (Female, Male) and Time (10 sec., 20 sec., 30 sec., 40 sec., 50 sec., and 60 sec.) were used as grouping variables; there were five levels of Task (Paradigms 1-5) and two levels of Artery (RMCA, LMCA). The last two factors are within-subject or repeated measures factors, because they represent repeated measurements of the effects of different tasks on MBFV in the RMCA and LMCA, on the same subject. The covariates were MBFV in Dark (RMCA, LMCA). The effect of each stimulus was assessed independently, and in comparison to the others. This was then followed by a planned contrast analysis comparing the effects of the each task relative to the other on the RMCA and LMCA in males and females respectively. All statistical calculations were performed using a statistical software package (Statistica, StatSoft, OK, USA).

Results

There was a significant main effect of Gender F (1,82)=8.2, p=0.005, MSe=982. There was a significant main effect of Paradigm F(4,336)=9.09, p=0.0000006, MSe=161. There were no main effects of Artery and Time. The effect of Paradigm was marginally modified by Time F(20,336)=1.57, p=0.058, MSe=27.8. The effect of Gender was modified by Artery F(1, 84)=6.2, p=0.1, MSe=1636.7. Similarly, the effect of Paradigm was modified by Artery F(4,336)=3.32, p=0.01, MSe =7.1. Consequently, there was a three way interaction of Gender×Paradigm×Artery F(4,336)=4.64, p=0.001, MSe=9.9.

TCD Spectroscopy

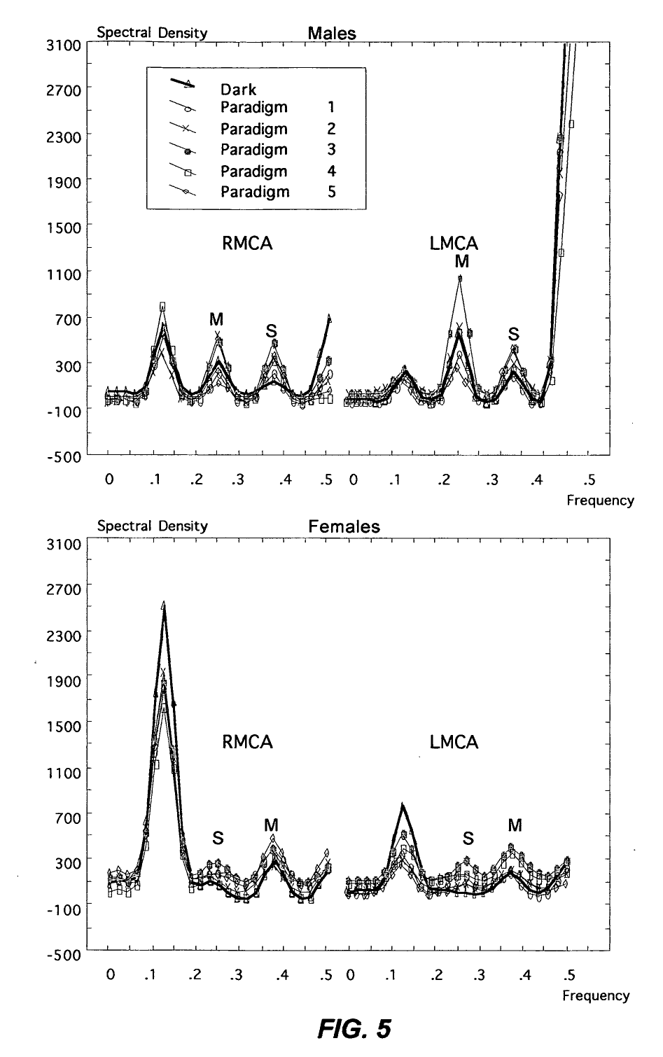

FIG. 5 shows the spectral density plots in male and female subjects in the RMCA and LMCA respectively. In males, in both RMCA and LMCA, the spectral density appeared to vary from right to left of the frequency bandwidth. The fundamental frequency related to the cardiovascular periodic oscillation occurred at a frequency of 0.5, and highest for dark, and was greater in the LMCA than RMCA (FIG. 4, top panel). At the frequency of 0.375, there was attenuation of the spectral density related to dark condition, more profoundly in the RMCA than LMCA. There was accentuation of spectral densities related to the other stimuli under light condition. This peak was thus designated as the stimulus spectral peak S. At S peak, the spectral density was highest for Paradigm 3 (facial memory task) 20. There was a higher and better separation of spectral densities at the S peak, in the RMCA compared to the LMCA. The next peak occurred at frequency of 0.25 and showed the spectral density related to dark condition mid-way between the other stimuli. This peak was designated as M peak, and may be related to mnemonic operations. There was a higher and better separation of the spectral densities at M peak, in the LMCA than RMCA. At M peak, the spectral density for Paradigm 3 20 was highest. The relationship of spectral densities for dark and stimuli in the next peak at frequency of 0.125, is similar to that seen for the fundamental frequency, and may thus reflect the hemodynamic characteristics of the cerebral vasculature. In females, in the RMCA and LMCA, the spectral density peaks appeared to vary from left to right of the frequency spectrum. The fundamental frequency occurred at 0.125 and was highest for dark, and greater in the RMCA than LMCA (FIG. 4, bottom panel). The S peak was identified at frequency of 0.27 with attenuation of the spectral density related to dark condition more profoundly in the LMCA than RMCA. There was accentuation of spectral densities related to stimuli, to higher levels from baseline in the LMCA than RMCA. The S peak was highest for Paradigm 3 (facial memory task) 20. The M peak occurred at frequency of 0.375.

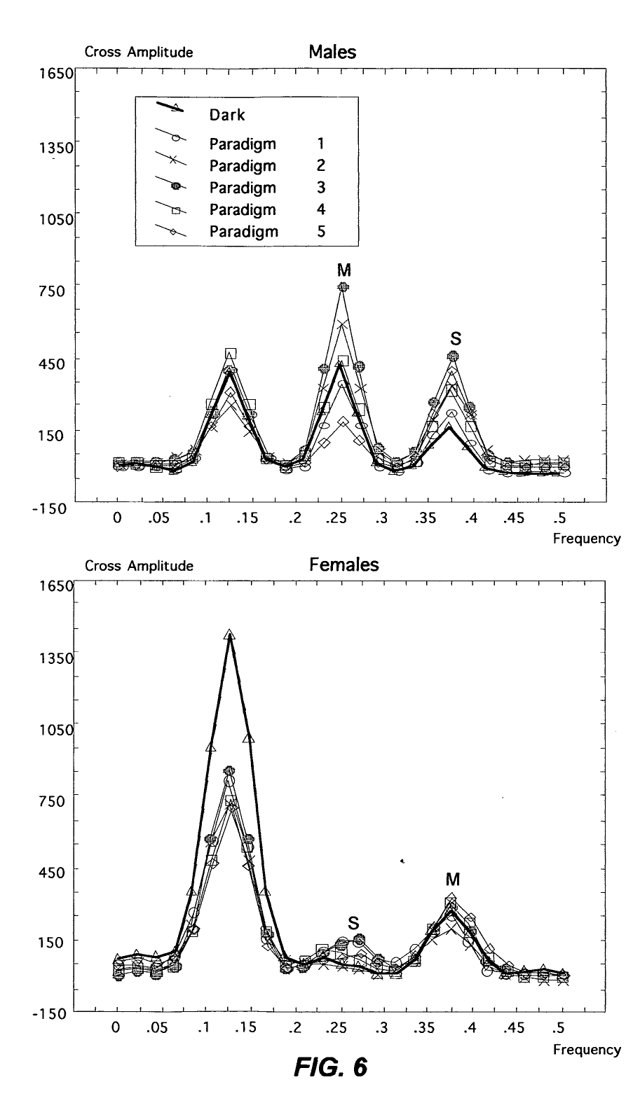

FIG. 6 shows the cross amplitude for the males and females respectively. The respective frequency components of the RMCA and LMCA, appeared to covary at S and M peaks in males (FIG. 6, top panel), more than females (FIG. 6, bottom panel).

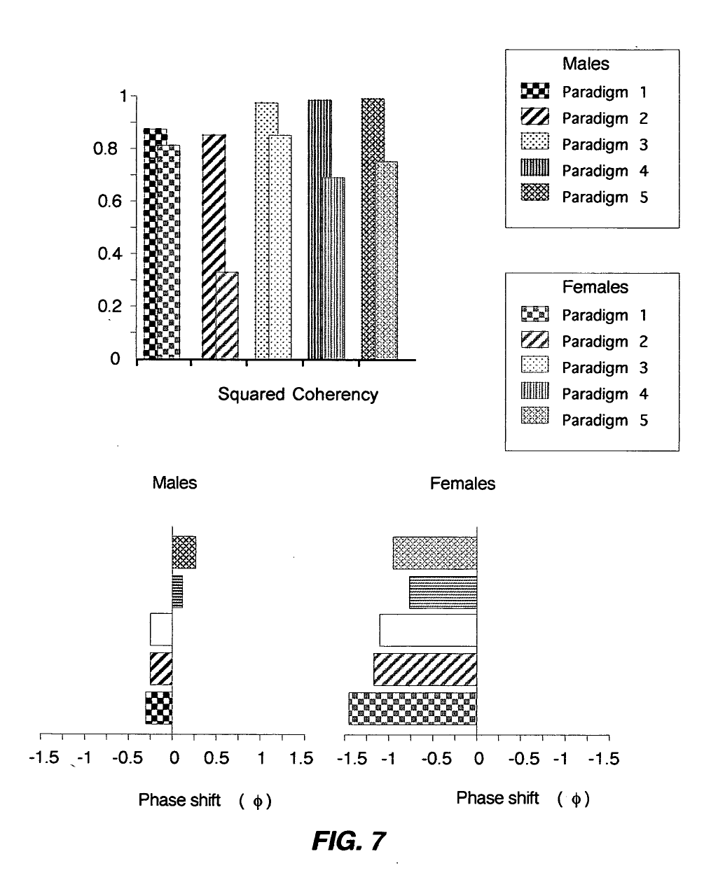

FIG. 7 shows the squared coherence plots in males and females subjects. In males, the squared coherence, showed a good measure (close to 1) of squared correlation between the cyclical components of the RMCA and LMCA at the S peak for all stimuli relative to dark (FIG. 7, top panel, back drop). In contrast, in females there was a relatively reduced squared coherence during stimuli, least during Paradigm 1 18 and highest for Paradigm 3 20 (FIG. 7, top panel, front). Phase shift analysis for S peak demonstrated that in male subjects, there was only a tendency towards left phase shift for Paradigms 1-3 18-20, and tendency towards right phase shift for Paradigms 4 21 and 5 22 (FIG. 7, bottom panel, left). In contrast, females had significant left phase lead for all stimuli (FIG. 7, bottom panel, right).

In conclusion, perception of faces requires iterative processes in the analysis of stimulus specific spectral characteristics. Frequency response of blood flow velocity changes suggest that, neuronal networks involved in processing of facial stimuli are attuned to respond to specific frequency bandwidths in a dynamic, self-organized, combinatorial and flexible fashion. In males, there was bi-hemispheric dynamic synchronization, while females utilized left intra-hemispheric mechanisms. The neuronal processing strategy used by females maybe more efficient in facial recognition tasks. On the other hand, detection of bihemispheric lateralization patterns in males but not females reveals retrieval processes associated with memory and implication of negative emotions.

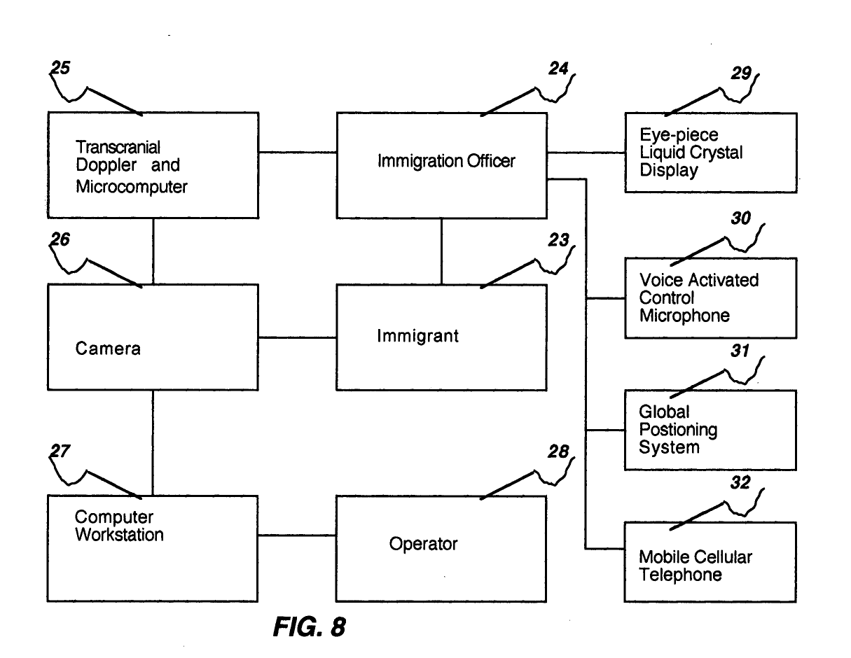

FIG. 8 shows the schematic diagram of one embodiment of the invention. In one embodiment of the present invention, an immigrant 23 presents him/herself to the immigration official 24, who is also a trained ‘face-minder’ interfaced with a transcranial Doppler device integrated in a pocket PC 25, such as that from Hewlett Packard Company, Palo Alto, Calif. by way of example. The immigration officer 24 examines the passport for routine border crossing check, at the same time, the immigrant is asked to pose for a passport photograph before a camera 26 during which time the immigration officer also focuses on the face of the immigrant simultaneously activating the acquisition of data by the transcranial Doppler device 25. The image acquired by the camera is exported to both the microcomputer operatively attached to the Transcranial Doppler 25, and the computer work station 27, where an operator 28 at the computer terminal stores, retrieves and matches the image to that in the biometric database. A number of accessories could become part of the personal carry-on devices that could be worn by the immigration officer or face-minder to help easy access to the database. A high resolution liquid crystal display (LCD) 29 could be used to visualize the faces and data displayed on the screen of the pocket PCD. Such LCD monitors adaptable for use in the present invention could be obtained at Olympus Optical Corporation GmbH, Hamburg, Germany. The control of the device could be made more convenient if most function keys could be voice activated using a microphone 30, attached to the headgear. In one embodiment of the invention, the immigration officer might be rooming along the border, in which case, a global positioning system (GPS) device 31 might be attached to the device to pinpoint his exact location. Such a GPS device is now an integral part of handheld PDAs and pocket PCs such as those from Hewlett Packard, Palo Alto Calif. Similarly, such devices are equipped with camera that could be used for the purpose of capturing the photograph of the face of the immigrant. In addition, the fingerprint data and personal records of criminal suspects could be stored as well in the pocket PC, especially for immigration officers that control long border crossings, such as that, between Mexico and the United States, by way of example. Illegal immigrants could be intercepted at different points along the border and the position of the immigration officer would be thus geographically referenced in real-time, and the photograph of the faces transmitted via a wireless mobile cellular telephone 32 (attached to the pocket PC), enabling cross matching with computer workstation database. Thereafter, a proper rapid deployment of resources to points of illegal entry along the border, could be achieved. In some cases, the database could be that of subjects without the necessary work permit, or those who have overstayed the stipulated visa duration rather than real criminals. The choice of application will vary with expected needs.

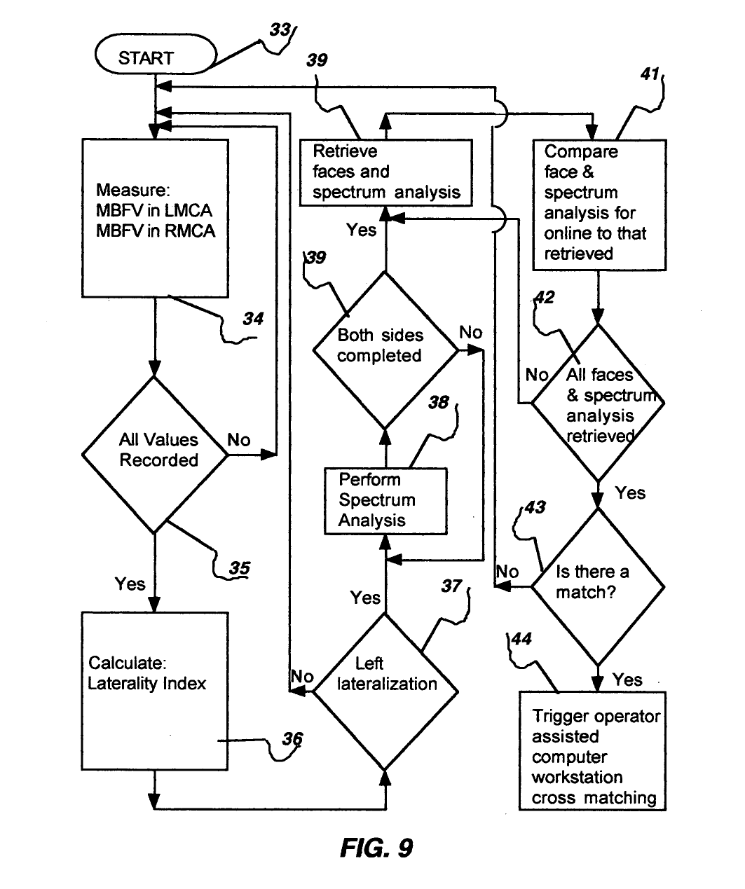

FIG. 9 shows the flow chart of one embodiment of the invention. In one embodiment of the invention, the immigration officer activates the ‘START button’ 33 of the transcranial Doppler device and microcomputer, for the acquisition of MBFV in the RMCA and LMCA 34, simultaneously while observing the subject's face in real-time. If not all values are recorded 35, the system repeats the step 34, but if all have been recorded, then the system proceeds to calculate the laterality index 36. It then determines if there is a left lateralization tendency 37, which may suggest that the face has been seen before probably from the facial biometric database viewed by the immigration officer as part of training or update exercise. The system uses the MBFV trend values to perform a spectrum analysis 38 for both RMCA and LMCA. If not all calculations are complete the system repeats the step 39. The system retrieves 40 the faces of criminal suspects and the corresponding spectral analysis for the faces as determined in the same immigration officer and ‘face minder’, by way of example. Each retrieved face and spectral analysis is compared to that in the database 41. All faces and their spectrum analysis are compared one after the other, until all is done 42, if not, the system repeats the step 41. The system searches for a match 43, if not present, the system repeats all of prior steps, but if present, the system triggers operator assisted computer workstation search 44. Based on the neuroscience, female operators might have better superiority in face recognition and could well cross-check identifications by their male counterparts who are best suited as face-minders. The perception that “I have seen that face before” evokes a peculiar brain response as disclosed by Sinha P and PoggioTI in an article entitled “I think I know that face” published in Nature, dated 1996, volume 348, page 404.

Several modifications of the present invention as described could be made depending on desired application. For example, the face could be replaced with the image of an objected intended for use in advertising. The desired spectrum analysis is stored in the database and the product image continually modified to yield the desired spectrum analysis response. Other applications could be illustrated with plastic and reconstructive surgery. The spectrum analysis of the desired facial attributes retrieved from a database could be used for comparison with the response evoked by the computer generated face intended for facial reconstruction. The device could be configured to provide facial, fingerprint, records, cerebral blood flow velocity data, and other biometric information in an integrated system or as combinations of selected functions. Other areas of application include the use of TCD spectroscopy for further analysis of data related to odor evaluabon as disclosed in U.S. Pat. No. 6,663,571 B1 to Njemanze; and application to define mental signature in mental performance testing systems as disclosed in U.S. Pat. No. 6,390,979 to Njemanze.

While a preferred embodiment of the present invention is described above, it is contemplated that numerous modifications maybe made thereto for particular applications without departing from the spirit and scope of the present invention. Accordingly, it is intended that the embodiment described be considered only as illustrative of the present invention and that the scope thereof should be limited thereto but be determined by reference to the claims hereinafter provided.

I claim:

1. A noninvasive method and system to determine cerebral blood flow velocity response to face recognition tasks in a human subject, including steps of:
(a) obtaining a subject's cerebral blood flow velocity in cerebral arteries on both sides of the brain using transcranial Doppler ultrasound instrument with two probes placed on the temples and sample volumes focused on cerebral vessels on both sides;
(b) simultaneously with (a) obtaining the mean blood flow velocity in both cerebral arteries at baseline;
(c) testing the subject on the screen of a digital computer and using a computer input peripheral device while simultaneously monitoring the mean blood flow velocity during each stage of the task in real-time;
(d) determining the response of the brain using mean cerebral blood flow velocity to a particular face retrieved from the database;
(e) determining side-to-side differences in mean cerebral blood flow velocity response to the face retrieved from the database;
(f) determining the spectrum analysis of the brain using the mean blood flow velocity response to the retrieved face;
(g) simultaneously with (f) obtaining the spectral density plots for both arteries;
(h) simultaneously with (g) identifying the respective frequency components for the left and right arteries;
(i) determining the brain response to the real face using laterality index and spectrum analysis of mean blood flow velocity oscillations;
j) determining the cross-amplitude as a measure of covariance between the respective frequency components in the two series determined for the retrieved face and real face for each artery respectively;
(k) simultaneously with j) determining the squared coherency as a measure of the squared correlation between the cyclical components in the two series at the respective frequency; and
(l) cross matching the brain response pattern to the real face as compared to that of the retrieved face.

2. The invention of claim 1 wherein the said device is operatively connected to a portable microcomputer that processes and displays the cerebral blood flow velocity, laterality index, faces, spectrum analysis, records, fingerprints and other biometric information in an integrated database or as a combination of selected data options.

3. The invention of claim 2 wherein the said device is operatively connected to a computer workstation for more extensive search and cross matching of faces to spectrum analysis and comparison by an operator.

4. The invention of claim 3 wherein the said device is a microcomputer operatively connected to a global positioning system.

5. The invention of claim 4 and further including a computer workstation means for retrieving the faces from an immigration, forensic, advertising or plastic surgery database.

6. The invention of claim 5 wherein the task involved relates to odor evaluation.

7. The invention of claim 5 wherein the task involved is a mental performance task and reflects the face-minder's perception of overall state-of-being of the immigrant.

8. The invention of claim 5 wherein the display of the microcomputer is operatively connected to an eye-piece monitor with optional voice control.

9. A noninvasive method and system to determine cerebral blood flow velocity response to face recognition tasks in a human subject, including steps of:
(a) obtaining a subject's cerebral blood flow velocity in cerebral arteries on both sides of the brain using transcranial Doppler ultrasound instrument with two probes placed on the temples and sample volumes focused on cerebral vessels on both sides;
b) simultaneously with (a) obtaining the mean blood flow velocity in both cerebral arteries at baseline;
(c) testing the male subject on the screen of a digital computer and using a computer input peripheral device while simultaneously monitoring the mean blood flow velocity during each stage of the task in real-time;
(d) determining the response of the brain using mean cerebral blood flow velocity to a particular face retrieved from the database;
(e) determining side-to-side differences in mean cerebral blood flow velocity response to the face retrieved from the database;
(f) determining the spectrum analysis of the brain using the mean blood flow velocity response to the retrieved face;
(g) simultaneously with (f) obtaining the spectral density plots for both arteries;
(h) simultaneously with (g) identifying the respective frequency components for the left and right arteries;
(i) determining the brain response to the real face using laterality index and spectrum analysis of mean blood flow velocity oscillations;
(j) determining the cross-amplitude as a measure of covariance between the respective frequency components in the two series determined for the retrieved face and real face for each artery respectively;
(k) simultaneously with (j) determining the squared coherency as a measure of the squared correlation between the cyclical components in the two series at the respective frequency; and
(l) determining if there is a match of the brain response pattern to the real face as compared to that of the retrieved face.

10. The invention of claim 9 wherein the said device is operatively connected to a portable microcomputer that processes and displays the cerebral blood flow velocity, laterality index, faces, spectrum analysis, personal records, fingerprints and other biometric information in an integrated database or as a combination of selected data options.

11. The invention of claim 10 wherein the said device is operatively connected to a computer workstation for more extensive search and cross matching of faces to spectrum analysis.

12. The invention of claim 10 wherein the said device is operatively connected to a computer workstation to trigger a more extensive search and cross matching of faces by a female operator.

13. The invention of claim 12 and further including a computer workstation means for retrieving the faces from a forensic or immigration biometric database.

14. The invention of claim 12 and further including a telecommunication means to connect to a computer workstation database.

15. A noninvasive method and system to determine cerebral blood flow velocity response to object recognition tasks in a human subject, including steps of:
(a) obtaining a subject's cerebral blood flow velocity in cerebral arteries on both sides of the brain using transcranial Doppler ultrasound instrument with two probes placed on the temples and sample volumes focused on cerebral vessels on both sides;
b) simultaneously with (a) obtaining the mean blood flow velocity in both cerebral arteries at baseline;
(c) testing the subject on the screen of a digital computer and using a computer input peripheral device while simultaneously monitoring the mean blood flow velocity during each stage of the task in real-time;
(d) determining the response of the brain using mean cerebral blood flow velocity to a particular image of an object retrieved from the database;
(e) determining the response of the brain using side-to-side differences in mean cerebral blood flow velocity response to the image of the object retrieved from the database;
(f) determining the spectrum analysis of the brain using the mean blood flow velocity response to the retrieved image of the object;
(g) simultaneously with (f) obtaining the spectral density plots for both arteries;
(h) simultaneously with (g) identifying the respective frequency components for the left and right arteries;
(i) determining the brain response to the real object using laterality index and spectrum analysis of mean blood flow velocity oscillations;
(j) determining the cross-amplitude as a measure of covariance between the respective frequency components in the two series determined for the retrieved image of the object and real object for each artery respectively;
(k) simultaneously with (j) determining the squared coherency as a measure of the squared correlation between the cyclical components in the two series at the respective frequency; and
(m) determining if there is a match of the brain response pattern to the real object as compared to that of the retrieved image of the object.

16. The invention of claim 15 wherein the said device is operatively connected to a portable microcomputer that processes and displays the cerebral blood flow velocity, laterality index, images of objects as well as the spectrum analysis in combination or as selected options.

17. The invention of claim 16 wherein the said device is operatively connected to a computer workstation for more extensive search and cross matching of the image of the object to spectrum analysis.

18. The invention of claim 17 wherein the image of the object under study comprise audiovisual scenes.

19. The invention of claim 17 wherein the object under study comprise odor specific characteristics.

20. The invention of claim 17 and further including a computer workstation means with human-computer interface system for object and facial recognition tasks for use in forensics, medicine and advertising.
